(19) United States
(12) Patent Application Publication
(10) Pub. No.: US 2024/0358408 A1
(43) Pub. Date: Oct. 31, 2024

(54) TRANSFER OF A PRETERM BABY FROM A NATURAL WOMB

(71) Applicant: TECHNISCHE UNIVERSITEIT EINDHOVEN, EINDHOVEN (NL)

(72) Inventors: Marlou MONINCX, EINDHOVEN (NL); Juliette Stephanie VAN HAREN, EINDHOVEN (NL); Franciscus Leonardus Marie DELBRESSINE, EINDHOVEN (NL); Daisy VAN DER WOUDE, EINDHOVEN (NL); Guid OEI, EINDHOVEN (NL)

(21) Appl. No.: 18/686,222

(22) PCT Filed: Aug. 23, 2022
(86) PCT No.: PCT/NL2022/050485

§ 371 Date: Feb. 23, 2024

(30) Foreign Application Priority Data
Aug. 23, 2021 (NL) 2029017

(51) Int. Cl. A61B 17/42; A61B 17/02; A61G 11/00

(52) U.S. Cl. CPC A61B 17/42; A61B 17/0293; A61G 11/00

(57) ABSTRACT

The present invention relates to a device for transferring a preterm from a natural womb of a pregnant mammal to an artificial womb, wherein the device comprises an adjustable first wound retractor and an adjustable second wound retractor, wherein each of the adjustable wound retractors is adapted to dilate a surgical wound incision to a desired diameter, comprising a flexible sleeve disposed in a cylindrical form between a first retraction ring and a second retraction ring, an annular shaped transfer device base placed over the second retraction rings, and a transfer bag device comprising a transfer bag having an opening which opening is connected to an annular shaped transfer device base coupling member, wherein the transfer bag is provided with at least one integrated glove for receiving the hand of a medical practitioner, in particular a gynaecologist, in order to allow the medical practitioner to transfer the preterm from the natural womb into the transfer bag.

### TECHNICAL FIELD

The present invention relates to a device for transferring a preterm from a natural womb of a pregnant mammal to an artificial womb, a kit-of-parts forming the device, a method for transferring a preterm from a natural womb of a pregnant mammal to an artificial womb as well as to a wound retractor, transfer base device and transfer bag device.

### BACKGROUND

Preterm birth, also known as premature birth, is the birth of a baby at fewer than 37 weeks gestational age, as opposed to full-term delivery at approximately 40 weeks. Very early preterm birth is before 32 weeks, early preterm birth occurs between 32-36 weeks, late preterm birth is between 34-36 weeks' gestation. The World Health Organization defines the following sub-categories of preterm birth, based on gestational age:
extremely preterm (less than 28 weeks);
very preterm (28 to 32 weeks); and
moderate to late preterm (32 to 37 weeks).

In line with the various regulations and directives throughout the world, there is a common understanding that induction or caesarean birth should not be planned before 39 completed weeks unless medically indicated as premature infants are at greater risk for cerebral palsy, delays in development, hearing problems and problems with their vision. The earlier a baby is born, the greater these risks will be.

However, despite this common understanding, spontaneous preterm birth may occur as well as the induction of labour or caesarean required for other medical reasons. There may be certain medical reasons for early delivery such as preeclampsia. Also, in view of the recent COVID-19 pandemic medical necessity for caesarean birth is more likely to occur in order to allow a pregnant woman to be intubated without having a direct negative effect on the foetus.

Preterm birth is the most common cause of death among infants worldwide. About 15 million babies are preterm each year (5% to 18% of all deliveries). Late preterm birth accounts for 75% of all preterm births. This rate is inconsistent across countries. In the United Kingdom 7.9% of babies are born pre-term and in the United States 12.3% of all births are before 37 weeks gestation. Approximately 0.5% of births are extremely early periviable births (20-25 weeks of gestation), and these account for most of the deaths. In many countries, rates of premature births have increased between the 1990s and 2010s. Complications from preterm births resulted in 0.81 million deaths in 2015, down from 1.57 million in 1990. The chance of survival at 22 weeks is about 6%, while at 23 weeks it is 26%, 24 weeks 55% and 25 weeks about 72%. The chances of survival without any long-term difficulties are lower.

Given the significant low survival rates of preterm birth, there is a need to develop procedures increasing the survival rates of preterm birth.

### DESCRIPTION OF THE INVENTION

In order to increase the survival rate of a preterm birth, in particular extremely preterm birth, the present invention provides hereto a device for transferring (also herein referred to as “transfer device”) a preterm baby from a natural womb of a pregnant mammal into a transfer bag. By providing such a transfer device, the preterm baby can be transferred, or transported by using the transfer bag, to an artificial womb to let the prematurely born child extend its stay. To let the child develop further in the artificial womb, a safe transfer from the natural womb to an artificial womb is now provided by the present invention.

It is stressed that the artificial womb and the transfer device according to the present invention for transferring the preterm baby to the artificial womb is intended to be used only in case of spontaneous preterm birth or induction of labour or caesarean required for other medical reasons in order to increase the survival rate of the preterm baby. By transferring a preterm baby from the natural womb to an artificial womb using the transfer device of the present invention, direct contact between the ambient environment (i.e. containing oxygen) and the preterm baby is herewith prevented. Thus optimizing and extending the ideal situation for the preterm baby, in particular for the lungs of the preterm baby, to further develop in a womb-like surrounding.

Although one could argue that by providing the transfer device according to the present invention and the eventual transfer of the preterm baby to an artificial womb, the transfer of the perinate from the natural womb to an artificial womb cannot qualify as giving birth. However, although the perinate is not in direct contact with the ambient environment (i.e. containing oxygen) and thus may (legally) considered not wholly brought into the world as such (as a self-breathing human being), in view of the present invention the transfer of the perinate from the natural womb classifies as giving birth (either via a caesarean section or via the birth canal). As a consequence, by transferring the perinate from the natural womb, the perinate classifies, according to the invention, as a preterm baby.

As used herein, the term “preterm baby” refers to the perinate (or foetus) comprised in the natural womb which needs to be delivered prematurely, i.e. before 37 weeks gestation, due to spontaneous (preterm) delivery or due to induced labour or caesarean section as required in case of a medical necessity. The term “preterm baby” further refers to any birth before 37 weeks gestation, also including extremely early periviable births (20-25 weeks gestation). Unless indicated otherwise, the term “preterm baby” includes extremely preterm (less than 28 weeks), very preterm (28 to 32 weeks), and moderate to late preterm (32 to 37 weeks). Other synonyms for the term “preterm baby” as used herein may include perinate or infant. Although not always correct, the term “preterm baby” may considered to be similar to the term “incubator baby”, i.e. an infant of which the vital organs needs to be developed further after birth.

As used herein, the term “artificial womb” may refer to a natural womb-mimicking device, wherein the preterm baby transferred from the natural womb may be develop further. In order to avoid any further interference, the umbilical cord of the preterm baby transferred into the artificial womb needs to be connected to an artificial placenta in order to allow (the vital organs of) the preterm baby to further develop. Synonyms for the term “artificial womb” include “preterm life support system”, “liquid based life support system” and “perinatal life support system”.

Also, as used herein, the term “pregnant mammal” may refer to a human being carrying a baby before giving birth to that baby. On the other hand, the term “pregnant mammal” may also refer to an animal, i.e. a mammal, wherein such animal is viviparous.

Further, as used herein, the terms “transferring” and “transfer” refer to the active step of transporting, moving, conveying or removing something from one place to another place. In the present invention the terms “transferring” and “transfer” refer to the movement of a preterm baby from its natural, original position in the natural womb of a pregnant mammal to another, in this case artificial, environment.

The present invention relates, in particular, to the transfer of a preterm baby from the natural womb to a transfer bag wherein the preterm baby is delivered via a caesarean section. In other words, the present invention does not relate to the transfer of a preterm baby from the natural womb to a transfer bag wherein the preterm baby is delivered via the birth canal.

In order to assist the transfer of a preterm baby from the natural womb to a transfer bag, wherein the preterm baby is delivered via a caesarean section, the present invention provides hereto a device for transferring a preterm from a natural womb of a pregnant mammal to an artificial womb, wherein the device comprises:
an adjustable first wound retractor and an adjustable second wound retractor, wherein each of the adjustable wound retractors is adapted to dilate a surgical wound incision to a desired diameter, the retractors comprising a flexible sleeve disposed in a cylindrical configuration between a first interior retraction ring and a second exterior retraction ring, wherein:
   the first retraction ring having a diameter greater than the desired diameter of the wound incision and being adapted to be disposed in the interior of the wound incision; and
   the second retraction ring having a central axis, an annular axis and a diameter greater than the desired diameter of the wound incision and being adapted to be disposed exterior of the wound incision, wherein the second retraction ring is rollable over itself and around the annular axis to provide the flexible sleeve with a radial retraction force sufficient to stretch the incision to the desired diameter,
an annular shaped transfer device base placed over the second retraction rings and releasably secured to at least one of the second retraction rings wherein the central axis of the transfer device base coincides with the central axis of the second retraction rings; and
a transfer bag device comprising a transfer bag having an opening which opening is connected to an annular shaped transfer device base coupling member, which transfer device base coupling member is placed over and releasably secured to the transfer device base.

Given the transfer device of the present invention, the transfer device base, the transfer bag or the transfer device base coupling member further comprises an inlet for supplying amniotic fluid, such as artificial amniotic fluid, to the interior of the transfer device base and/or the transfer bag device. Further, the transfer bag of the transfer device of the present invention is provided with at least one integrated glove for receiving the hand of a medical practitioner, in particular a gynaecologist, in order to allow the medical practitioner to transfer the preterm from the natural womb into the transfer bag.

By providing the transfer device of the present invention, a smooth and seamless transfer of the preterm baby from the natural womb into the transfer bag can be guaranteed wherein any involvement of ambient conditions during delivery is prevented. In other words, the present invention provides for an assisted delivery via the caesarean section wherein the preterm baby transferred from the natural womb does not come into contact with ambient conditions, such as oxygen containing air. Even further, by providing the transfer device of the present invention, the preterm baby will not be influenced by external factors, such as change in light intensity, ambient air pressure, temperature fluctuations of the (artificial) amniotic fluid, and the like.

The different parts of the transfer device to assist the transfer of a preterm baby from the natural womb, via a caesarean section, to an artificial environment, such as a transfer bag are further described below.

### Adjustable First and Second Wound Retractors

In order to have the transfer device to assist the transfer of a preterm baby from the natural womb, via the caesarean section, to an artificial environment the transfer device of the present invention comprises first and second wound retractors in order to avoid any interaction between the preterm baby and the incisions made in order to provide the caesarean section and in order to provide a connection between the natural womb and the artificial environment, such as a transfer bag which connection is able to contain (artificial) amniotic fluid. In order to provide such a protective connection, the wound retractors of the present invention comprises a flexible sleeve disposed in a cylindrical configuration between a first interior retraction ring and a second exterior retraction ring.

The adjustable first wound retractor is preferably selected from known wound retractors, such as an Alexis® retractor or an SurgiSleeve™. As the adjustable first wound retractor is used at the laparotomy site of the caesarean section, the first wound retractor is preferably configured for use at the laparotomy site.

The adjustable second wound retractor, is preferably configured for use at the hysterotomy site. In a preferred embodiment of the present invention, the adjustable second wound retractor is an uterus retractor.

It is noted that the adjustable second wound retractor deviates from the wound retractors known in the art in that the diameter of the first interior retraction ring is smaller than the diameter of the second exterior retraction ring. Therefore, in a second aspect of the present invention the invention relates to an adjustable wound retractor adapted to dilate a surgical wound incision, such as a wound incision at the hysterotomy site, to a desired diameter, comprising a flexible sleeve disposed in a cylindrical form between a first retraction ring and a second retraction ring, wherein:
the first retraction ring having a diameter greater than the desired diameter of the wound incision and being adapted to be disposed interior of the wound incision; and
the second retraction ring having an annular axis and a diameter greater than the desired diameter of the wound incision and being adapted to be disposed exterior of the wound incision, wherein the second retraction ring is rollable over itself and around the annular axis to provide the flexible sleeve with a radial retraction force sufficient to stretch the incision to the desired diameter, wherein the diameter of the first retraction ring is smaller than the diameter of the second retraction ring.

Further to the above, the second retraction rings of one of the wound retractors or each of the wound retractors is preferably configured to connect to the annular shaped transfer device base. In order to provide such a connection the second retraction rings of one of the or both wound retractors may be made from a thickening material. Such thickening material may be similar to the material used for the retraction rings of currently known wound retractors (such as the Alexis® retractor or the SurgiSleeve™). The second retraction rings may cooperate with a circumferential groove provided in the transfer base device to provide a reliable and water-tight sealed connection between one or both retractors and the transfer base device.

Regarding the flexible sleeve of the wound retractor, it is noted that the length of such a flexible sleeve may be similar to the length of the flexible sleeve of known wound retractors (such as the Alexis® retractor or the SurgiSleeve™).

### Annular Shaped Transfer Device Base

The transfer device of the present invention comprises a transfer device base in order to connect the wound retractors to a transfer bag device comprising a transfer bag. The transfer device base of the present invention is provided with an annular shaped pass-through opening, wherein the pass-through opening is configured to allow the preterm baby to pass through the base device. Although the dimensions of such pass-through opening should be chosen such that the preterm baby is allowed to pass through, it is further noted that the dimensions of the pass-through opening should be selected such that the hand of a medical practitioner is also able to pass-through the opening. As such the diameter of the pass-through opening is preferably selected in the range of 10 cm to 20 cm.

As already provided above, the transfer device base may be configured to receive the second exterior retraction ring of one or both of the wound retractors. In an embodiment of the present invention, the transfer base device may be provided with receiving means, such as a circumferential groove, for receiving a second exterior retraction ring of one of the wound retractors.

However, in order to provide the most solid and reliable connection between the transfer device base and the first and second wound retractors of the transfer device of the present invention, the present invention relates in a third aspect to a transfer device base for use in a transfer device according to the present invention, wherein the transfer device base is annular shaped and wherein the transfer device base is provided with at least two circumferential grooves wherein each of the circumferential grooves is configured to receive a respective second retraction ring.

In order to releasably secure the wound retractors and/or the transfer bag device to the transfer device base of the present invention, the transfer device base may be provided with securing means. Suitable securing means may include a bayonet connection, threaded connection, or clip connection. In addition to the suitable securing means, other sealing means may be present in the transfer device base. Suitable sealing means may include a circumferential flexible O-rings and/or a circumferential labyrinth closure, preferably provided in a recess of the transfer device base.

Although the transfer device base may have any suitable form, an annular form similar to the annular form of the pass-through opening provided in the transfer device base is preferred. In addition, the transfer device base is preferably rigid in order to provide a reliable and solid connection between the transfer device base and the wound retractors and transfer bag device.

### Transfer Bag Device

The transfer device of the present invention, further comprises a transfer bag device comprising a transfer bag for receiving the preterm baby transferred from the natural womb. The transfer bag of the present invention is a flexible bag provided with an opening, preferably an annular shaped receiving opening. The dimensions of the receiving opening are chosen such as to allow the preterm baby to be received in the transfer bag. In addition the volume of the transfer bag may be selected such as to mimic the volume of the natural womb wherefrom the preterm baby is transferred. Preferred volumes of the transfer bag are within the range of 800 mL to 3000 mL.

The transfer bag of the present invention is provided with at least one integrated glove for receiving the hand of a medical practitioner, in particular a gynaecologist, in order to allow the medical practitioner to access the natural womb and to allow facilitated transfer of the preterm baby from the natural womb into the transfer bag. Preferably the integrated glove is positioned opposite to the opening provided in the transfer bag.

The opening of the transfer bag of the present invention is further provided with (connected to) an annular shaped transfer device base coupling member for releasably coupling the transfer bag to the transfer device base. In a preferred embodiment the coupling member is an annular shaped coupling member corresponding to the annular shaped receiving opening of the transfer bag. Even further, the coupling member may be in the form of a solid rigid ring for providing a secured and reliable coupling between the transfer device and the base device. It is noted that the dimensions of the receiving opening are preferably chosen such that they closely resembles the dimensions of the pass-through opening provided in the base body of the base device, i.e. in the range of between 10 cm to 20 cm.

The transfer bag of the present invention may further be provided with an inlet for supplying a liquid, such as an artificial amniotic fluid (AAF), to the interior of the transfer bag. Obviously, during use, by providing a liquid to the interior of the transfer bag, the supply of the liquid is automatically extended to the interior of the transfer device base and the wound retractors. Further, the transfer bag may further be provided with a valve, e.g. an air vent, for releasing air from the transfer bag. Such release of air via a valve is highly preferred in case the transfer device of the present invention is supplied with liquid. In use, the transfer device comprises one main opening, i.e. the opening facing the natural womb formed by the wound retractors. Pressure build-up in the transfer device (by supplying liquid to the transfer device) will negatively affect the pressure in the natural womb in case no air valve is provided.

In a further embodiment, the transfer bag may be provided with an integrated closure, such as an integrated wire, to close the transfer bag after the preterm baby is transferred from the natural womb into the transfer bag. In order to avoid accidental squeezing of the umbilical cord of the preterm baby, the closure of the receiving opening of the transfer bag is facilitated by loosely securing the integrated closure around the umbilical cord connected to the preterm baby. Preferably the integrated closure is arranged in the vicinity of the opening provided in the transfer bag.

Once the baby is received in the transfer bag, the transfer device may be released from the transfer device base. In order to further process the transfer of the preterm baby to a further artificial environment, such as an artificial womb, the transfer bag is preferably placed on a horizontal plane such that the receiving opening of the transfer bag is facing upwards away from the horizontal plane.

As provided above, the transfer bag device according to the present invention is providing a secure and reliable connection between the wound retractors, such that the preterm can be transferred into the transfer bag comprised by the transfer bag device in a safe and secure manner. In this respect, the present invention relates in a fourth aspect of the present invention to a transfer bag device for transferring a preterm, wherein the transfer bag device comprises a transfer bag having an opening which opening is connected to an annular shaped transfer device base coupling member, which transfer device base coupling member is configured to be placed over and releasably secured to a transfer device base, wherein the transfer bag is provided with at least one integrated glove for receiving the hand of a medical practitioner, in particular a gynaecologist, in order to allow the medical practitioner to transfer the preterm from the natural womb into the transfer bag.

It is noted that according to this fourth aspect of the present invention, the transfer bag or the transfer device base coupling member further comprises an inlet for supplying amniotic fluid, such as artificial amniotic fluid, to the interior of the transfer bag device. In a further embodiment of this fourth aspect of the present invention, the integrated glove is positioned opposite to the opening provided in the transfer bag of the transfer bag device. As already disclosed above, in this fourth aspect of the present invention, the transfer bag of the transfer bag device may further comprise an integrated closure, such as an integrated wire, to close the transfer bag after the preterm is transferred from the natural womb into the transfer bag.

### Aspects of the Present Invention

In addition to the four aspects of the present invention, i.e. the transfer device of the present invention, the adjustable wound retractor, the transfer device base and the transfer bag device, as provided above, the present invention further relates to a fifth, sixth and seventh aspect.

In the fifth aspect of the present invention, the present invention relates to a kit-of-parts for forming a device for transferring a preterm from a natural womb of a pregnant mammal to an artificial womb, the kit-of-parts comprising:
an adjustable second wound retractor adapted to dilate a surgical wound incision at the hysterotomy site to a desired diameter, comprising a flexible sleeve disposed in a cylindrical form between a first retraction ring and a second retraction ring, wherein:
   the first retraction ring having a diameter greater than the desired diameter of the wound incision at the hysterotomy site and being adapted for disposition interiorly of the wound incision; and
   the second retraction ring having a central axis, an annular axis and a diameter greater than the desired diameter of the wound incision and being adapted for disposition exteriorly of the wound incision, wherein the second retraction ring is rollable over itself and around the annular axis to provide the flexible sleeve with a radial retraction force sufficient to stretch the incision to the desired diameter,
an annular shaped transfer device base configured to be placed over and releasably secured to the second retraction ring such that the central axis of the transfer device base coincides with the central axis of the second retraction rings; and
a transfer bag device comprising a transfer bag having an opening which opening is connected to an annular shaped transfer device base coupling member, which transfer device base coupling member is configured to be placed over and releasably secured to the transfer device base.

The transfer device base, the transfer bag or the transfer device base coupling member of the kit-of-parts of the present invention further comprises an inlet for supplying amniotic fluid, such as artificial amniotic fluid, to the interior of the transfer device base and/or the transfer bag device. Additionally, the transfer bag is provided with at least one integrated glove for receiving the hand of a medical practitioner, in particular a gynaecologist, in order to allow the medical practitioner, in use, to transfer the preterm from the natural womb into the transfer bag.

The kit-of-parts according to the present invention may further comprise an adjustable first wound retractor adapted to dilate a surgical wound incision at the laparotomy site to a desired diameter, comprising a flexible sleeve disposed in a cylindrical form between a first retraction ring and a second retraction ring, wherein:
the first retraction ring having a diameter greater than the desired diameter of the wound incision at the laparotomy site and being adapted for disposition interiorly of the wound incision; and
the second retraction ring having a central axis, an annular axis and a diameter greater than the desired diameter of the wound incision and being adapted for disposition exteriorly of the wound incision, wherein the second retraction ring is rollable over itself and around the annular axis to provide the flexible sleeve with a radial retraction force sufficient to stretch the incision to the desired diameter.

In a sixth aspect of the present invention, the invention relates to a method for transferring a preterm baby from a natural womb of a pregnant mammal to an artificial womb, the method comprising the steps of:
a) providing the kit-of-parts of claim 14;
b) providing a pregnant mammal, wherein the pregnant mammal is provided with a first wound retractor having a first retraction ring placed interiorly of a wound incision at the laparotomy site and having a second retraction ring placed exteriorly of the wound incision, and wherein the pregnant mammal is further provided with a wound incision at the hysterotomy site;
c) inserting the first retraction ring of the second wound retractor into the wound incision at the hysterotomy site in order to place the first retraction ring interiorly of the wound incision and having the second retraction ring of the second wound retractor placed exteriorly of the wound incision;
d) placing the transfer device base over the second retraction rings and releasably securing the transfer device base to at least one of the second retraction rings;
e) placing the transfer bag device over the transfer device base and releasably securing the transfer device base coupling member of the transfer bag device to the transfer device base in order to allow the medical practitioner, in particular a gynaecologist, to transfer the preterm from the natural womb into the transfer bag; and
f) before allowing the medical practitioner, in particular a gynaecologist, to transfer the preterm from the natural womb into the transfer bag, supplying amniotic fluid, such as artificial amniotic fluid, to the interior of the transfer device base.

In a seventh aspect of the present invention, the invention relates to a method for transferring a preterm baby from a natural womb of a pregnant mammal to an artificial womb, the method comprising the steps of:
i) providing an pregnant mammal;
ii) providing a wound incision at the laparotomy site in the pregnant mammal provided in step i);
iii) inserting a first retraction ring of a first wound retractor into the wound incision at the laparotomy site provided in step ii) in order to place the first retraction ring interiorly of the wound incision and having a second retraction ring of the first wound retractor placed exteriorly of the wound incision;
iv) providing a wound incision at the hysterotomy site in the pregnant mammal provided in step i);
v) inserting a first retraction ring of a second wound retractor into the wound incision at the hysterotomy site in order to place the first retraction ring interiorly of the wound incision and having a second retraction ring of the second wound retractor placed exteriorly of the wound incision;
vi) placing a transfer device base over the second retraction rings and releasably securing the transfer device base to at least one of the second retraction rings;
vii) placing a transfer bag device comprising a transfer bag having an opening and an integrated glove, which opening is connected to an annular shaped transfer device base coupling member, over the transfer device base and releasably securing the transfer device base coupling member of the transfer bag device to the transfer device base;
viii) supplying amniotic fluid, such as artificial amniotic fluid, to the interior of the transfer bag device;
ix) allowing a medical practitioner, in particular a gynaecologist, to access the natural womb by hand using the integrated glove provided in the transfer bag; and
x) transferring the preterm from the natural womb into the transfer bag.

Further to the methods of the sixth and seventh aspects of the present invention, the transfer bag may comprise an integrated closure and wherein the methods further comprises the step of:
closing the transfer bag by loosely securing the integrated closure of the transfer bag around the umbilical cord connected to the preterm.

As a final step of the methods according to the sixth and seventh aspects of the present invention, the method may further comprise the step of:
releasing the transfer device base from the second retraction rings and turning the transfer bag connected to the transfer device base upside down, i.e. by turning the assembly of the transfer bag and transfer device by about 180º.

### EXAMPLES

The present invention will be further described by way of example only with reference to the accompanying drawings, in which:

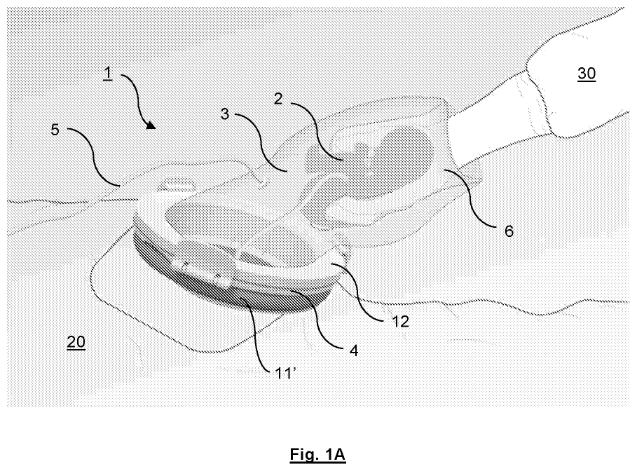

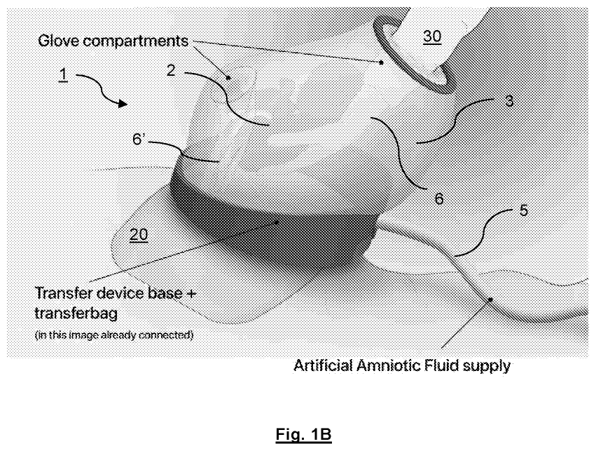

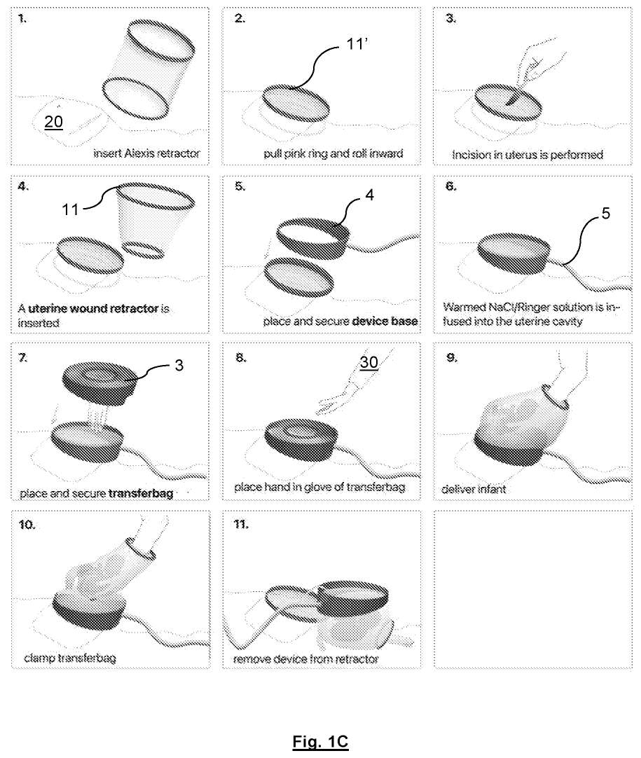

FIG. 1A shows a transfer device according to the first aspect of the invention.

FIG. 1B shows an alternative transfer device according to the first aspect of the invention.

FIG. 1C shows the method of transferring a preterm baby from the natural womb into the transfer bag according to the sixth and seventh aspect of the invention.

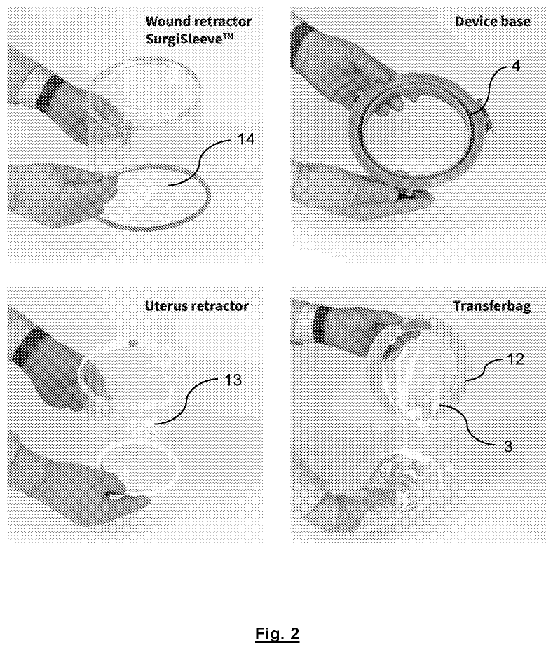

FIG. 2 shows the kit-of-parts according to the fifth aspect of the invention.

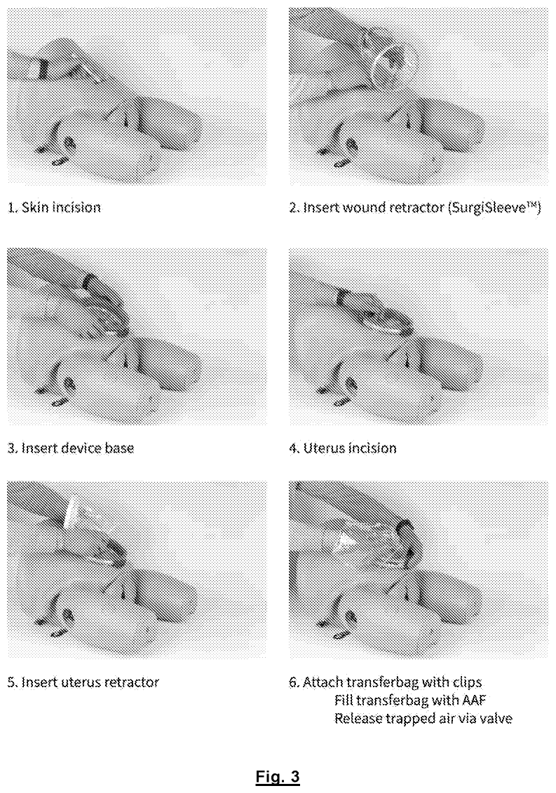

FIG. 3 shows the method of transferring a preterm baby from the natural womb into the transfer bag according to the sixth and seventh aspect of the invention.

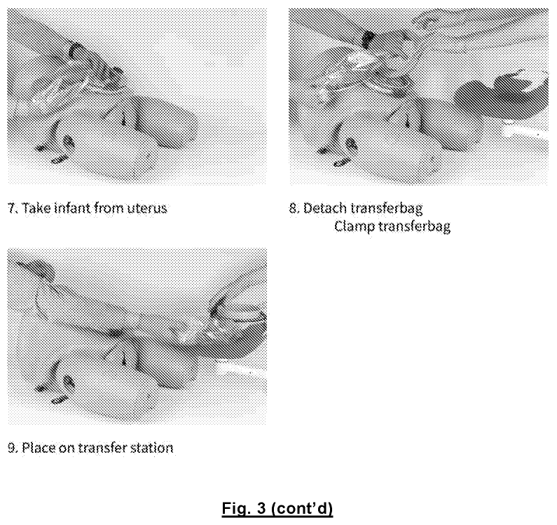

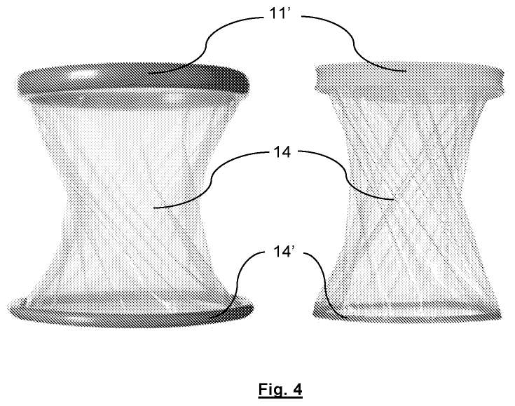

FIG. 4 shows the wound retractors used in the transfer device according to the first aspect of the invention.

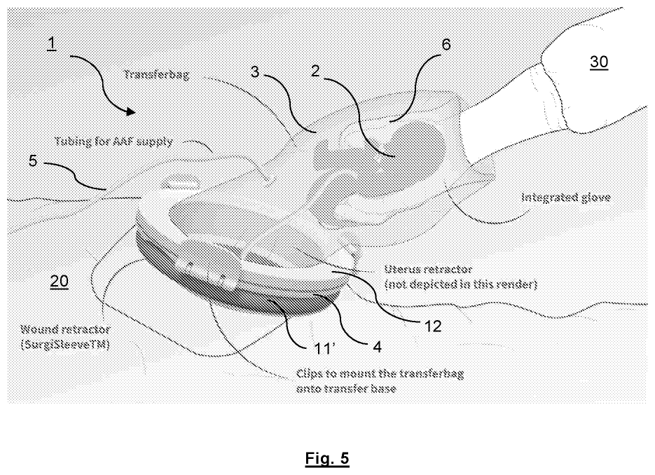

FIG. 5 shows the transfer device according to the first aspect of the invention.

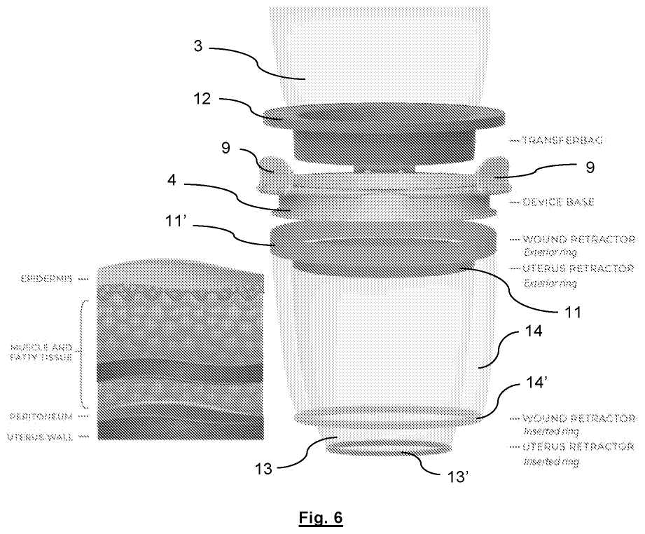

FIG. 6 shows an exploded view of the assembly of the transfer device according to the first aspect of the invention.

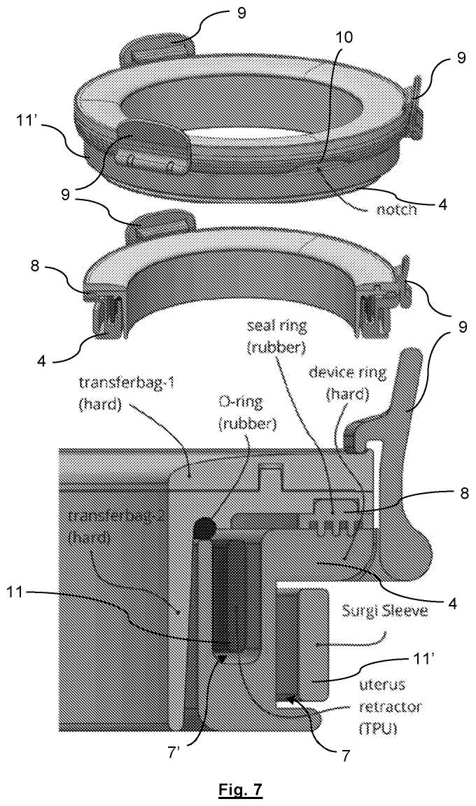

FIG. 7 shows a detailed part of the transfer device base according to the fourth aspect of the invention.

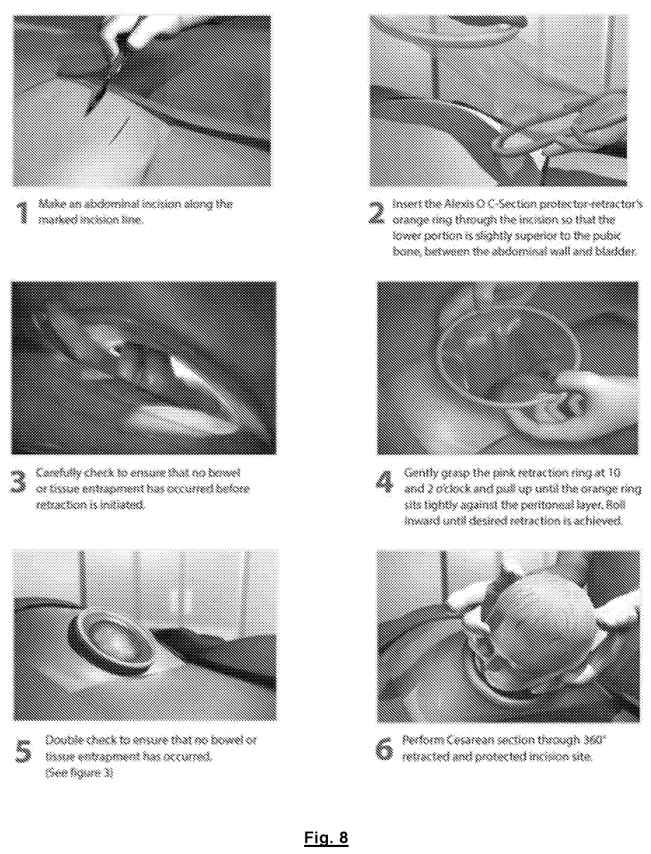

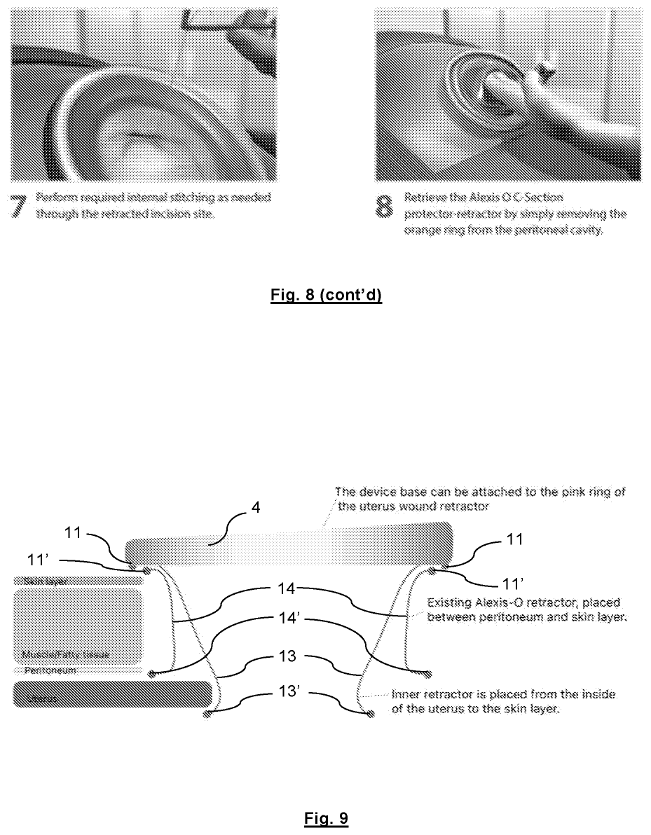

FIG. 8 shows the standard caesarean section procedure.

FIG. 9 is a cross-sectional view of the wound retractors and the transfer device base.

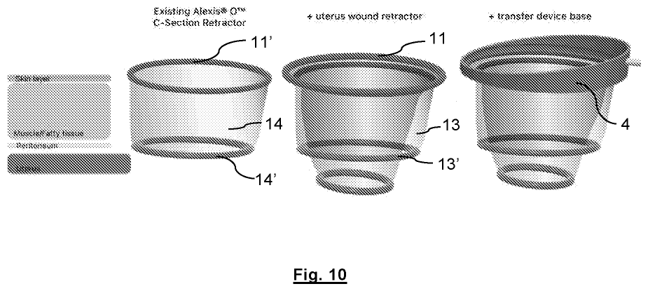

FIG. 10 shows the method of placing the wound retractors and transfer device base.

FIG. 1A and FIG. 5 show a transfer device 1 according to the present invention which can be used to safely transfer a preterm 2 (premature infant) from the natural womb (not shown in FIG. 1A) into a transfer bag 3. The transfer device 1 can be integrated into the current caesarean section procedure. To this end a Pfannenstiel incision is made up to the uterus, after which a wound retractor is inserted to maintain a stable opening. For this, use is made of an existing retractor: the Alexis® O C-section protector/retractor (from Applied Medical) or the SurgiSleeve™ Wound Protector (from Medtronic), shown in FIG. 4. Subsequently, a uterus incision, hysterotomy, is performed. After this, a uterus wound retractor is placed from the skin to the inner lining of the uterus to function as a tampon to control bleeding, to prevent blood accumulation in the transfer bag, to prevent Artificial Amniotic Fluid flow into the maternal abdominal cavity and finally to prevent infant-air contact. These wound retractors form a structure onto which the transfer device base 4 and transfer bag 3 can be attached.

The transfer bag 3 is placed onto the transfer device base 4. AAF (Artificial Amniotic Fluid) is supplied, via supply 5, and access to the preterm 2 can be given through the integrated glove 6. The preterm 2 is delivered after which the transfer bag 3 can be placed and stabilized in horizontal position and detached from the transfer device base 4.

In an alternative embodiment (FIG. 1B), the transfer device base 4 is placed over the outer exterior retraction ring (not shown) and a small volume of warmed Artificial Amniotic Fluid (AAF) is supplied via AAF supply 5. The transfer bag 3 is placed over the transfer device base 4. AAF is supplied and access to the preterm 2 can be given through the integrated gloves 6, 6′. The preterm 2 is delivered after which the transfer bag 3 can be placed and stabilized in horizontal position and detached from the transfer device base 4.

A more detailed overview of the method of transferring a preterm baby from the natural womb into the transfer bag is provided in FIG. 3. The method is explained in further detail below.

In case the pregnant mammal 20 (hereinafter referred to as ‘the candidate’) complies with the required criteria for a C-section transfer procedure she will be prepared for surgery. The candidate 20 is placed in a supine tilted position to avoid aortocaval compression that can occur when the uterus compresses the aorta, inferior vena cava (IVC), or both. During the preparation phase, the abdomen will be cleaned with an antiseptic and sterile drapes will be placed to create a sterile field and to block the patient's vision.

After the preparation and after determining that the administered anesthesia is considered effective, the gynecologist 30 makes a Pfannenstiel incision through the skin and fascia of 14 cm in width (FIG. 3, item 1). During this stage, a procedural adjustment must be made to safely perform the transfer procedure. A crucial requirement is that the preterm is kept submerged in fluid to prevent gas exchange. The protocol will continue closely resembling the normal C-section procedure (FIG. 8) until the laparotomy is performed. When reaching the uterus a wound retractor such as the Alexis® retractor or SurgiSleeve™ (FIG. 4) can be used by inserting the interior ring through the incision so that the lower portion is slightly superior to the pubic bone, between the abdominal wall and bladder (FIG. 3, item 2). After the gynecologist carefully checked to ensure that no bowel or tissue entrapment has occurred, the exterior retraction ring is pulled up until the interior ring is tight against the peritoneal layer. The exterior retraction ring is grasped under a 120° angle between both hands and rolled inward (see also: FIG. 8, item 4) until the desired retraction is achieved. Next, the device base is placed and ‘clamped’ inside the exterior ring of the wound retractor (the wound retractor neatly fits into the device base) (FIG. 3, item 3). As can be seen in FIG. 7, the exterior ring of the wound retractor fits into a groove 7 in the transfer device base 4.

The uterus is now exposed and a hysterotomy can be performed (FIG. 3, item 4). Subsequently a second wound retractor, the uterus wound retractor, is placed (FIG. 3, item 5), of which the smaller interior ring is inserted (could be in a folded state) into the uterus and the exterior ring is pulled up and rolled inward until the desired retraction is achieved. The exterior ring can then be placed in the designated slot or groove 7′ in the transfer device base 4 as depicted in FIG. 7. The design of the uterus retractor is based on the above-mentioned wound retractor, yet consists of a smaller interior ring which fits inside the uterus and can be placed towards the inner lining of the uterus wall. This double retractor combination aims to prevent AAF from entering into the maternal abdominal cavity as it creates a barrier between the two exterior rings, of the uterus and wound retractor. This design choice reduces the risk of TUR syndrome, prevents blood accumulation in the transfer bag, creates a closed channel between the natural and artificial environment, and the desired retraction can be achieved with both retractors.

The gynaecologist 30 may now place a hand inside the glove of the transfer bag 3. Before the transfer bag 3 is placed onto the transfer device base 4, it is already filled for a large portion with AAF. When access to the preterm 2 is confirmed, the transfer bag 3 can be placed onto the transfer device base 4 as depicted in FIG. 7 (see also: FIG. 3, item 6). Rubber seals 8 (with labyrinth grooves) are placed at strategic places to ensure watertight seals (see FIG. 7). Three clips 9 will secure the transfer bag 3 onto the device base 4 by creating pressure, a reversible action when the preterm 2 has been delivered.

The AAF supply 5, 5′ can be turned on to fill an additional amount of fluid, trapped air can be released through a valve (not shown) integrated in the transfer bag (FIG. 3, item 6).

Upon delivery of the preterm 2, the gynaecologist has easy access through the integrated glove 6 (FIG. 3, item 7).

When the preterm 2 is fully transferred into the transfer bag 3, an additional person clamps the transfer bag 3 by hand to avoid leakage of AAF, the transfer bag 3 needs to be detached from the base 4 by lifting the bag 3 from the device base 4, aided through the integration of notches 10 as depicted in FIG. 7. Subsequently, the bag 3 can be turned for 180° to stabilize the entire transfer bag 3 next to the mother since the umbilical cord is still attached to the placenta (see FIG. 3.8). The transfer bag will be supported by a transfer station, that can be placed on or next to the mother 20 (FIG. 3, item 9). When the transfer towards the artificial womb (AW) is successfully performed the umbilical cord will be cannulated to connect it to the artificial placenta (FIG. 3, item 9).

In an alternative embodiment (FIG. 1C), the transfer device base 4 can be placed over the exterior placed retraction rings 11, 11′. A tap, as used in the GelPort® from Applied Medical, can be slide underneath the pink retraction ring 11′ to ensure it is in place. Similar to the GelPort®, this layer can have a latch that needs to be pressed to create resistance to secure the layer. The transfer device base 4 must be pulled up gently to ensure that it is in place and secured (FIG. 1C, item 5). The AAF supply 5′ can be turned on to fill the external space and the peritoneal cavity (FIG. 1C, item 6). When access to the preterm 2 is confirmed, the transfer bag 3 can be placed over the transfer device base 4 (FIG. 1C, item 7). After the transfer bag 3 is in place, the latch can be closed to secure. The transfer bag must be pulled up gently to ensure that it is in place and secured. The AAF supply 5′ is activated and the gynaecologist can place a hand inside the glove 6 (FIG. 1C, item 8). By pulling the arm upwards the transfer bag 3 will fold out and can be filled with AAF. When desired the other hand can be placed in the integrated glove 6′ in the transfer bag 3. When the preterm 2 completely enters the transfer bag 3 the gynaecologist can give support using their hands (FIG. 1C, item 9). An additional person clamps the transfer bag 3 by pulling the integrated wire until it is loosely secured around the umbilical cord and the transfer bag 3 can be detached (FIG. 1C, item 10). Subsequently, it can be turned for 180° to stabilize the entire transfer bag 3 next to the mother 20 since the umbilical cord is still attached to the placenta. The transfer bag 3 will be supported by a transfer station, that can be placed on or next to the mother 20.

When the transfer towards the AW is successfully performed the umbilical cord will be cannulated to connect it to the artificial placenta (FIG. 1C, item 11).

FIG. 2 shows the kit-of-parts according to the fifth aspect of the present invention, comprising the transfer device base 4 and the transfer bag 3 provided with coupling means 12 for securing the transfer bag 3 to the transfer device base 4. The kit-of-parts further comprises a wound retractor 13, functioning as an uterus retractor. The kit-of-parts may further comprise a standard wound retractor 14, such as the SurgiSleeve™.

FIG. 6 shows an exploded view of the assembly of the transfer device 1 of the present invention further in relation to the placement of the different parts of the transfer device 1 in view of the pregnant mammal 20 body. The transfer device 1 comprises a first wound retractor 14 having an interior ring 14′ and an exterior ring 11′. Additionally, the transfer device 1 comprises a second wound retractor 13, having an interior ring 13′ and an exterior ring 11. Both exterior rings 11, 11′ are connectable to the transfer device base 4, here depicted with clips 9 to releasably secure the coupling member 12 of the transfer bag 3. In addition, as shown in FIG. 6, the interior ring 14′ of the first wound retractor 14 is placed against the peritoneal layer, whereas the interior ring 13′ of the second wound retractor 13 is placed the inner lining of the uterus wall. FIG. 9 is a more schematical cross-sectional view of the placement of the interior rings 13′, 14′ and exterior rings 11, 11′ in connection to the transfer device base 4, whereas FIG. 10 is a 3D view of FIG. 9 including the first and second wound retractors 13, 14 and the transfer device base 4.

1. Device for transferring a preterm from a natural womb of a pregnant mammal to an artificial womb, wherein the device comprises:
an adjustable first wound retractor and an adjustable second wound retractor, wherein each of the adjustable wound retractors is adapted to dilate a surgical wound incision to a desired diameter, the retractors comprising a flexible sleeve disposed in a cylindrical configuration between a first interior retraction ring and a second exterior retraction ring, wherein:
the first retraction ring having a diameter greater than the desired diameter of the wound incision and being adapted to be disposed in the interior of the wound incision; and
the second retraction ring having a central axis, an annular axis and a diameter greater than the desired diameter of the wound incision and being adapted to be disposed exterior of the wound incision, wherein the second retraction ring is rollable over itself and around the annular axis to provide the flexible sleeve with a radial retraction force sufficient to stretch the incision to the desired diameter,
an annular shaped transfer device base placed over the second retraction rings and releasably secured to at least one of the second retraction rings wherein the central axis of the transfer device base coincides with the central axis of the second retraction rings; and
a transfer bag device comprising a transfer bag having an opening which opening is connected to an annular shaped transfer device base coupling member, which transfer device base coupling member is placed over and releasably secured to the transfer device base,
wherein the transfer device base, the transfer bag or the transfer device base coupling member further comprises an inlet for supplying amniotic fluid, such as artificial amniotic fluid, to the interior of the transfer device base and/or the transfer bag device, and
wherein the transfer bag is provided with at least one integrated glove for receiving the hand of a medical practitioner, in particular a gynaecologist, in order to allow the medical practitioner to transfer the preterm from the natural womb into the transfer bag.

2. Device according to claim 1, wherein the integrated glove is positioned opposite to the opening provided in the transfer bag of the transfer bag device.

3. Device according to claim 1, wherein the transfer bag of the transfer bag device further comprises an integrated closure, such as an integrated wire, to close the transfer bag after the preterm is transferred from the natural womb into the transfer bag.

4. Device according to claim 3, wherein the integrated closure is configured such that the closure of the transfer bag is facilitated by loosely securing the integrated closure around the umbilical cord connected to the preterm.

5. Device according to claim 1, wherein the adjustable first wound retractor is configured for use at the laparotomy site and wherein the adjustable second wound retractor is configured for use at the hysterotomy site.

6. Device according to claim 1, wherein the adjustable first wound retractor is a wound retractor preferably selected from Alexis® retractor or an SurgiSleeve™.

7. Device according to claim 1, wherein the adjustable second wound retractor is an uterus retractor.

8. Adjustable wound retractor adapted to dilate a surgical wound incision, such as a wound incision at the hysterotomy site, to a desired diameter, comprising a flexible sleeve disposed in a cylindrical form between a first retraction ring and a second retraction ring, wherein:
the first retraction ring having a diameter greater than the desired diameter of the wound incision and being adapted to be disposed interior of the wound incision; and
the second retraction ring having an annular axis and a diameter greater than the desired diameter of the wound incision and being adapted to be disposed exterior of the wound incision, wherein the second retraction ring is rollable over itself and around the annular axis to provide the flexible sleeve with a radial retraction force sufficient to stretch the incision to the desired diameter,
wherein the diameter of the first retraction ring is smaller than the diameter of the second retraction ring.

9. Transfer device base for use in a device according to claim 1, wherein the transfer device base is annular shaped and wherein the transfer device base is provided with at least two circumferential grooves wherein each of the circumferential grooves is configured to receive a respective second retraction ring.

10. Transfer bag device for transferring a preterm, wherein the transfer bag device comprises a transfer bag having an opening which opening is connected to an annular shaped transfer device base coupling member, which transfer device base coupling member is configured to be placed over and releasably secured to a transfer device base of claim 9, wherein the transfer bag is provided with at least one integrated glove for receiving the hand of a medical practitioner, in particular a gynaecologist, in order to allow the medical practitioner to transfer the preterm from the natural womb into the transfer bag.

11. Transfer bag device according to claim 10, wherein the transfer bag or the transfer device base coupling member further comprises an inlet for supplying amniotic fluid, such as artificial amniotic fluid, to the interior of the transfer bag device.

12. Transfer bag device according to claim 10, wherein the integrated glove is positioned opposite to the opening provided in the transfer bag of the transfer bag device.

13. Transfer bag device according to claim 10, wherein the transfer bag of the transfer bag device further comprises an integrated closure, such as an integrated wire, to close the transfer bag after the preterm is transferred from the natural womb into the transfer bag.

14. Kit-of-parts for forming a device for transferring a preterm from a natural womb of a pregnant mammal to an artificial womb, the kit-of-parts comprising:
an adjustable second wound retractor adapted to dilate a surgical wound incision at the hysterotomy site to a desired diameter, comprising a flexible sleeve disposed in a cylindrical form between a first retraction ring and a second retraction ring, wherein:
the first retraction ring having a diameter greater than the desired diameter of the wound incision at the hysterotomy site and being adapted for disposition interiorly of the wound incision; and
the second retraction ring having a central axis, an annular axis and a diameter greater than the desired diameter of the wound incision and being adapted for disposition exteriorly of the wound incision, wherein the second retraction ring is rollable over itself and around the annular axis to provide the flexible sleeve with a radial retraction force sufficient to stretch the incision to the desired diameter,
an annular shaped transfer device base configured to be placed over and releasably secured to the second retraction ring such that the central axis of the transfer device base coincides with the central axis of the second retraction rings; and
a transfer bag device comprising a transfer bag having an opening which opening is connected to an annular shaped transfer device base coupling member, which transfer device base coupling member is configured to be placed over and releasably secured to the transfer device base,
wherein the transfer device base, the transfer bag or the transfer device base coupling member further comprises an inlet for supplying amniotic fluid, such as artificial amniotic fluid, to the interior of the transfer device base and/or the transfer bag device, and
wherein the transfer bag is provided with at least one integrated glove for receiving the hand of a medical practitioner, in particular a gynaecologist, in order to allow the medical practitioner, in use, to transfer the preterm from the natural womb into the transfer bag.

15. Kit-of-parts according to claim 14, wherein the kit-of-parts further comprises an adjustable first wound retractor adapted to dilate a surgical wound incision at the laparotomy site to a desired diameter, comprising a flexible sleeve disposed in a cylindrical form between a first retraction ring and a second retraction ring, wherein:
the first retraction ring having a diameter greater than the desired diameter of the wound incision at the laparotomy site and being adapted for disposition interiorly of the wound incision; and
the second retraction ring having a central axis, an annular axis and a diameter greater than the desired diameter of the wound incision and being adapted for disposition exteriorly of the wound incision, wherein the second retraction ring is rollable over itself and around the annular axis to provide the flexible sleeve with a radial retraction force sufficient to stretch the incision to the desired diameter.

16. Method for transferring a preterm from a natural womb of a pregnant mammal to an artificial womb, the method comprising the steps of:
a) providing the kit-of-parts of claim 14;
b) providing a pregnant mammal, wherein the pregnant mammal is provided with a first wound retractor having a first retraction ring placed interiorly of a wound incision at the laparotomy site and having a second retraction ring placed exteriorly of the wound incision, and wherein the pregnant mammal is further provided with a wound incision at the hysterotomy site;
c) inserting the first retraction ring of the second wound retractor into the wound incision at the hysterotomy site in order to place the first retraction ring interiorly of the wound incision and having the second retraction ring of the second wound retractor placed exteriorly of the wound incision;
d) placing the transfer device base over the second retraction rings and releasably securing the transfer device base to at least one of the second retraction rings;
e) placing the transfer bag device over the transfer device base and releasably securing the transfer device base coupling member of the transfer bag device to the transfer device base in order to allow the medical practitioner, in particular a gynaecologist, to transfer the preterm from the natural womb into the transfer bag; and
f) before allowing the medical practitioner, in particular a gynaecologist, to transfer the preterm from the natural womb into the transfer bag, supplying amniotic fluid, such as artificial amniotic fluid, to the interior of the transfer device base.

17. Method for transferring a preterm from a natural womb of a pregnant mammal to an artificial womb, the method comprising the steps of:
i) providing an pregnant mammal;
ii) providing a wound incision at the laparotomy site in the pregnant mammal provided in step i);
iii) inserting a first retraction ring of a first wound retractor into the wound incision at the laparotomy site provided in step ii) in order to place the first retraction ring interiorly of the wound incision and having a second retraction ring of the first wound retractor placed exteriorly of the wound incision;
iv) providing a wound incision at the hysterotomy site in the pregnant mammal provided in step i);
v) inserting a first retraction ring of a second wound retractor into the wound incision at the hysterotomy site in order to place the first retraction ring interiorly of the wound incision and having a second retraction ring of the second wound retractor placed exteriorly of the wound incision;
vi) placing a transfer device base over the second retraction rings and releasably securing the transfer device base to at least one of the second retraction rings;
vii) placing a transfer bag device comprising a transfer bag having an opening and an integrated glove, which opening is connected to an annular shaped transfer device base coupling member, over the transfer device base and releasably securing the transfer device base coupling member of the transfer bag device to the transfer device base;
viii) supplying amniotic fluid, such as artificial amniotic fluid, to the interior of the transfer bag device;
ix) allowing a medical practitioner, in particular a gynaecologist, to access the natural womb by hand using the integrated glove provided in the transfer bag; and
x) transferring the preterm from the natural womb into the transfer bag.

18. Method according to claim 16, wherein the transfer bag comprises an integrated closure and wherein the method further comprises the step of:
closing the transfer bag by loosely securing the integrated closure of the transfer bag around the umbilical cord connected to the preterm.

19. Method according to claim 16, wherein the method further comprises the step of:
releasing the transfer device base from the second retraction rings and turning the transfer bag connected to the transfer device base upside down, i.e. by turning the assembly of the transfer bag and transfer device by about 180°.
